(19) United States
(12) Patent Application Publication
(10) Pub. No.: US 2021/0198327 A1
(43) Pub. Date: Jul. 1, 2021

(54) SUMO AND USES THEREOF

(71) Applicant: PLICO BIOTECH INC., New York, NY (US)

(72) Inventors: Luca COLNAGHI, Cornate d'Adda, Monza E Brianza (IT); Luana FIORITI, Seveso, Monza E Brianza (IT); Amir LEVINE, New York, NY (US)

(21) Appl. No.: 16/087,548

(22) PCT Filed: Mar. 22, 2017
(86) PCT No.: PCT/IB2017/051667

§ 371 Date: Sep. 21, 2018

(30) Foreign Application Priority Data
Mar. 24, 2016 (IT) 102016000031116

(51) Int. Cl. C07K 14/47; A61P 25/28; A61K 45/06; A61K 38/17

(52) U.S. Cl. CPC C07K 14/47; A61P 25/28; C07K 14/473; C07K 2319/00; A61K 38/1709; A61K 38/1741; A61K 45/06

(57) ABSTRACT

A protein that is at least one SUMO protein, or a variant or a fragment thereof or a fusion protein including a SUMO protein is for use in the treatment of neurodegenerative and/or neurological disorders. A pharmaceutical composition includes a protein that is at least one SUMO protein or a variant or a fragment thereof or a fusion protein including a SUMO protein and pharmaceutically acceptable carriers.

It is here described a protein which is at least one SUMO protein, or a variant or a fragment thereof or a fusion protein comprising the same for use in the treatment of neurodegenerative and/or neurological disorders. In a further embodiment, it is here described a pharmaceutical composition comprising a protein which is at least one SUMO protein or a variant or a fragment thereof or a fusion protein comprising the same and pharmaceutically acceptable carriers.

### BACKGROUND

Neurodegenerative disorders are a class of debilitating conditions characterized by a progressive impairment of cognitive or motor functions. Although these disorders present a range of symptoms in patients, they share a number of similarities at the cellular and molecular levels. The most prominent one is neuronal loss, likely caused by the formation of toxic protein aggregates. Biochemical analyses have identified several proteins in these aggregates, with tau and alpha-synuclein being the most represented in samples derived from Alzheimer's and Parkinson's patients respectively, linking the two proteins to the diseases.

SUMO (Small Ubiquitin-like Modifier) is a small protein that can be covalently attached to target proteins (J. R. Gareau, C. D. Lima, The SUMO pathway: emerging mechanisms that shape specificity, conjugation and recognition. Nature reviews Molecular cell biology 2010; 11, 861). SUMO has been implicated in a large series of cellular processes, from cell signaling to DNA repair, and it has also been suggested that its attachment to a protein helps the targeted protein to remain soluble (R. Grana-Montes, et al. N-terminal protein tails act as aggregation protective entropic bristles: the SUMO case. Biomacromolecules 2014; 15, 10 1194). The role of SUMO in modulating aggregation is still controversial and opposite hypotheses have been formulated and proposed regarding the role of SUMO in either promoting or inhibiting aggregation. There are 4 confirmed SUMO isoforms in humans: SUMO1, SUMO2, SUMO3 and SUMO4. SUMO2/3 show a high degree of similarity to each other and are distinct from SUMO1. SUMO4 shows similarity to SUMO2/3 but differs in having a Proline instead of Glutamine at position 90. As a result, SUMO4 is not processed and conjugated under normal conditions; SUMO4 is used for modification of proteins under stress-conditions like starvation (W. Wei, et al. A stress-dependent SUMO4 SUMOylation of its substrate proteins. Biochem Biophys Res Commun 2008; 375 (3): 454-459). In the four isoforms, SUMO is obtained from immature SUMO with the cleaving off of a propeptide at the C-terminus, leaving a C-terminal glycine residue on SUMO.

In the past decade, it has been suggested that a series of post-translational modifications (PTMs) modulates aberrant aggregation. For instance, both tau and alpha-synuclein are hyper phosphorylated when aggregated, suggesting that phosphorylation could be a positive signal for aggregation. The two proteins are also modified by the proteins SUMO1 and SUMO2/3 (V. Dorval, P. E. Fraser Small ubiquitin-like modifier (SUMO) modification of natively unfolded proteins tau and alpha-synuclein. JBC 2006; 281, 9919).

The present invention addresses the strong need for novel and effective therapeutic treatment for neurodegenerative and/or neurological diseases.

### DESCRIPTION

Here it is firstly disclosed the use of SUMO or variants thereof in preventing/treating neurodegeneration. SUMO derivatives capable to pass through the blood brain barrier and to reach the CNS are here disclosed. Said derivatives are here demonstrated useful in preventing and treating neurodegenerative conditions.

### FIGURES DESCRIPTION

FIG. 1: SUMO2 immature form (SEQ ID no. 16) and SUMO2 (SEQ ID no. 1). The proteases cleavage site is indicated by an arrow.

FIG. 2: schematic representation of an embodiment of the present invention, where a SUMO immature protein linked to a carrier via a linker is used.

FIG. 3: schematic representation of a further embodiment of the present invention, where three SUMO, at least the first one in the immature form, linked together via a linker are used.

FIG. 4: SUMO2 (SEQ ID no. 1) in vitro activity versus tau-induced toxicity.

FIG. 5: SUMO2 (SEQ ID no. 1), SUMO2 immature form (SEQ ID no. 7) and SUMO2 immature form poly gene (3X)—Transferrin peptide (SEQ ID no. 8) in vitro activity versus tau-induced toxicity.

FIG. 6: SUMO2 (SEQ ID no. 1), SUMO2 immature form (SEQ ID no. 7) and SUMO2 immature form poly gene (3X)—Transferrin peptide (SEQ ID no. 8) in vitro activity versus alpha-synuclein-induced toxicity.

FIG. 7: SUMO2 (SEQ ID no. 1), SUMO2 immature form (SEQ ID no. 7) and SUMO2 immature form poly gene (3X)—Transferrin peptide (SEQ ID no. 8) in vitro activity versus HTT-induced toxicity.

FIG. 8: Blood brain barrier penetrance of SUMO2 (SEQ ID no. 1) and SUMO2 immature form (SEQ ID no. 7).

### DETAILED DESCRIPTION

It has been here firstly demonstrated SUMO activity in preventing toxicity induced by aggregated protein. The results, described in the experimental section below, clearly demonstrate that SUMO is not toxic for the cells and it is capable to rescue from protein aggregates-induced toxicity.

Proteins selected from the group comprising SUMO, or immature SUMO, preferably immature SUMO2, or a variant or a fragment thereof or fusion proteins comprising SUMO, or immature SUMO, preferably immature SUMO2, or a variant or a fragment thereof, for use in the treatment of neurodegenerative and/or neurological diseases are here described.

Fusion proteins comprising SUMO, capable to pass through the blood brain barrier and to reach the CNS, are here disclosed. Said derivatives are here demonstrated useful in preventing and treating neurodegenerative and/or neurological conditions. Generally, when an amino acid sequence of the invention (or a compound, construct or fusion protein comprising the same) is intended for administration to a subject (for example, for therapeutic purposes as described herein), it is preferably either an amino acid sequence that does not occur naturally in said subject or, when it does occur naturally in said subject, it is in essentially isolated form (as defined herein).

For the aim of the present description, when referring to “SUMO” it is intended any one of the SUMO isoforms: SUMO1, SUMO2, SUMO3, SUMO4, in the immature or in the mature form.

In an embodiment, the here described proteins and fusion proteins are useful in the treatment of neurodegenerative disorders characterized by an aberrant protein aggregation, preferably selected from the group comprising: Alzheimer's Disease (AD), Parkinson's Disease (PD), Prion Disease, Amyotrophic Lateral Sclerosis (ALS), Spinocerebellar Ataxia Type 1, 3, 6 or 7 (SCAT, SCA3, SCA6, SCAT), Huntington's Disease (HD), Dentatorubral-Pallidoluysian Atrophy (DRPLA), Spinal and Bulbar Muscular Atrophy (SBMA).

In a first embodiment, said SUMO protein is a SUMO isoform selected from the group consisting of: SUMO1 immature form (SEQ ID no. 20), SUMO2 immature form (SEQ ID no. 16), SUMO3 immature form (SEQ ID no. 21), SUMO4 immature form (SEQ ID no. 22), SUMO1 (SEQ ID no. 23), SUMO2 (SEQ ID no. 1), SUMO3 (SEQ ID no. 24), SUMO4 (SEQ ID no. 25), or it is a SUMO mutant selected from SUMO2 K11A (SEQ ID no. 14), SUMO2 Q90P (SEQ ID no. 15) or it is a SUMO variant having a sequence at least 80%, or at least 90%, or at least 95%, or at least 98%, or at least 99% identical to any one of said sequences: SEQ ID no. 20, SEQ ID no. 21, SEQ ID no. 22, SEQ ID no. 23, SEQ ID no. 24, SEQ ID no. 25, SEQ ID no. 1, SEQ ID no. 16, SEQ ID no. 14, SEQ ID no. 15.

SUMO1 immature form (SEQ ID no. 20):

MSDQEAKPSTEDLGDKKEGEYIKLKVIGQDSSEIHFKV
KMTTHLKKLKESYCQRQGVPMNSLRFLFEGQRIADNHT
PKELGMEEEDVIEVYQEQTGGHSTV

SUMO3 immature form (SEQ ID no. 21):

MSEEKPKEGVKTENDHINLKVAGQDGSVVQFKIKRHTP
LSKLMKAYCERQGLSMRQIRFRFDGQPINETDTPAQLE
MEDEDTIDVFQQQTGGVPESSLAGHSF

SUMO4 immature form (SEQ ID no. 22):

MANEKPTEEV KTENNNHINL KVAGQDGSVV QFKIKRQTPL
SKLMKAYCEP RGLSMKQIRF RFGGQPISGT DKPAQLEMED
EDTIDVFQQP TGGVY

SUMO1 (SEQ ID no. 23):

MSDQEAKPSTEDLGDKKEGEYIKLKVIGQDSSEIHFKVK
MTTHLKKLKESYCQRQGVPMNSLRFLFEGQRIADNHTPK
ELGMEEEDVIEVYQEQTGG

SUMO3 (SEQ ID no. 24):

MSEEKPKEGVKTENDHINLKVAGQDGSVVQFKIKRHTPL
SKLMKAYCERQGLSMRQIRFRFDGQPINETDTPAQLEME
DEDTIDVFQQQTGG

SUMO4 (SEQ ID no. 25):

MANEKPTEEV KTENNNHINL KVAGQDGSVV QFKIKRQTPL
SKLMKAYCEP RGLSMKQIRF RFGGQPISGT
DKPAQLEMEDEDTIDVFQQP TGG

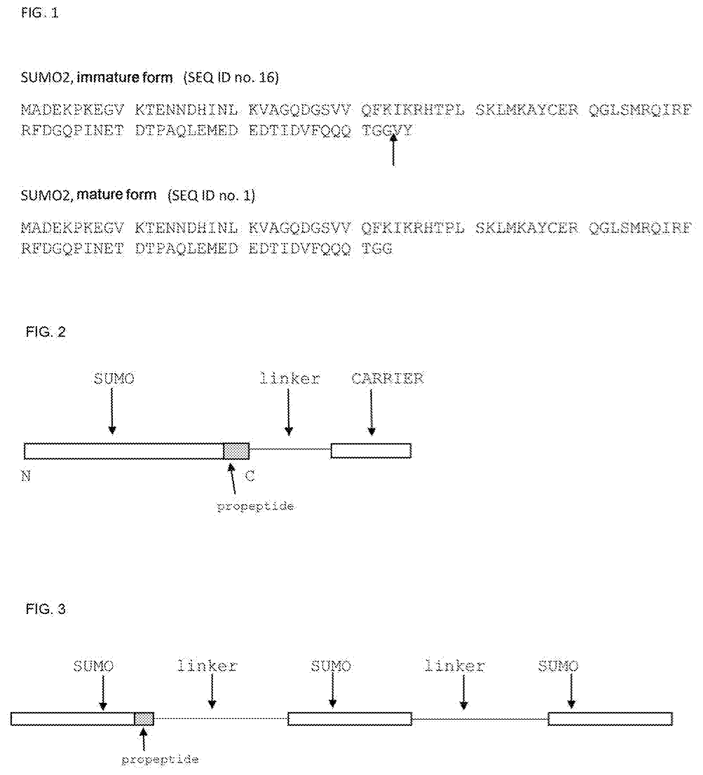

In a preferred embodiment, said SUMO protein, immature form, is linked to a carrier, wherein the linking between SUMO and the carrier is at the SUMO C-terminus, preferably via a linker, according to FIG. 2. The proteases that cleave off the SUMO propeptide are present in all the cells in the intracellular space, while they are not present in the intercellular spaces. Therefore, said fusion protein “immature SUMO-carrier” is maintained until said fusion protein enters into a cell. Once entered the intracellular space, the proteases cleave off at the C-terminus of immature SUMO, cleaving the propeptide together with the carrier and leaving SUMO available. Preferably, said immature SUMO is SUMO2, immature form (SEQ ID no. 16) and said mature form is therefore SUMO2 (SEQ ID No. 1), depicted in FIG. 1.

In a further preferred embodiment, said carrier is preferably selected in the group comprising: peptide linking the transferrin receptor, apolipoprotein B (apoB), (LRP-1/2) Angiopep-1, (LRP-1/2) Angiopep-2, (LRP-1/2) Angiopep-3, Rabies Virus Glycoprotein 29 (RVG29).

Where said carrier is linked to immature SUMO via a linker, said linker is any short amino acid sequence. Preferably, said linker is selected in the group comprising the following amino acidic sequences: AA, (GGGG)n where n indicates that said sequence GGGG (SEQ ID no. 17) is repeated at least once in said linker, (GGGGS)n where n indicates that said sequence GGGGS (SEQ ID no. 18) is repeated at least once in said linker, more preferably is repeated three times: GGGGSGGGGSGGGGS (SEQ ID no. 19).

In a still more preferred embodiment, said fusion protein is selected from the group comprising:

SUMO2 immature form—transferrin peptide (SEQ ID no. 2):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA CRTIGPSVC

SUMO2 immature form—LDLR-binding domain of ApoB (SEQ ID no. 3):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA SSVIDALQYK LEGTTRLTRK
RGLKLATALS LSNKFVEGS

SUMO2 immature form—(LRP-1/2) Angiopep-1(SEQ ID no. 4):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA TFFYGGCRGKRNNFKTEEY

SUMO2 immature form—(LRP-1/2) Angiopep-2(SEQ ID no. 5):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA TFFYGGSRGKRNNFKTEEY

SUMO2 immature form—(LRP-1/2) Angiopep-3 (SEQ ID no. 6):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA TFFYGGSRGKRNNFRTEEY

SUMO2 immature form—the neuron-specific rabies viral glycoprotein (RVG29) peptide (SEQ ID no. 7):

MADEKPKEGV KTENNDHINL KVAGQDGSVV
QFKIKRHTPL SKLMKAYCER QGLSMRQIRF
RFDGQPINET DTPAQLEMED EDTIDVFQQQ
TGGVY AA YTIWMPENPRPGTPCDIFTNSRGKRASNG

In a second embodiment, said fusion protein comprises at least two SUMO proteins linked together. Preferably, 3 SUMO proteins are linked together, according to FIG. 3. Preferably, a linker is inserted between the at least two SUMO, at least said first SUMO being in the immature form, said linker being formed by any short amino acid sequence, preferably said linker being independently selected among the following amino acidic sequences: AA, (GGGG)n where n indicates that said sequence GGGG (SEQ ID no. 17) is repeated at least once in said linker, (GGGGS)n where n indicates that said sequence GGGGS (SEQ ID no. 18) is repeated at least once in said linker, more preferably is repeated three times: GGGGSGGGGSGGGGS (SEQ ID no. 19). The at least two, preferably three SUMO are linked together via a C-terminus—N-terminus linkage. The obtained fusion protein facilitates the entrance in the CNS and in the intracellular space. Once inside, the proteases cleave off the SUMOs after the first SUMO immature form at the cleavage site to cleave off the propeptide, allowing mature SUMO to be present in the intracellular space. FIG. 3 is a schematic representation of an embodiment where 3 SUMO are linked together via a linker. In a preferred embodiment, said fusion protein comprises two or more SUMO2 proteins, at least the first of them being SUMO2 immature form. More preferably, the two or more SUMO are further linked to a carrier. In a preferred embodiment, said fusion protein is selected from:

SUMO2 immature form poly gene (3X)—Transferrin peptide (SEQ ID no. 8):

MADEKPKEGV KTENNDHINL KVAGQDGSVV QFKIKRHTPL
SKLMKAYCER QGLSMRQIRF RFDGQPINET DTPAQLEMED
EDTIDVFQQQTGGVY AA MADEKPKEGV KTENNDHINL
KVAGQDGSVV QFKIKRHTPL SKLMKAYCERQGLSMRQIRF
RFDGQPINET DTPAQLEMED EDTIDVFQQQ TGGVY
AAMADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA CRTIGPSVC

SUMO2 immature form poly gene (3X)—LDLR-binding domain of ApoB (SEQ ID no. 9):

MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA MADEKPKEGV KTENNDHINL
KVAGQDGSVV QFKIKRHTPL SKLMKAYCER QGLSMRQIRF
RFDGQPINET DTPAQLEMED EDTIDVFQQQ TGGVY AA
MADEKPKEGV KTENNDHINL KVAGQDGSVV QFKIKRHTPL
SKLMKAYCERQGLSMRQIRF RFDGQPINET DTPAQLEMED
EDTIDVFQQQ TGGVY AA SSVIDALQYK LEGTTRLTRK
RGLKLATALS LSNKFVEGS

SUMO2 immature form poly gene (3X)—(LRP-1/2) Angiopep-1 (SEQ ID no. 10):

MADEKPKEGV KTENNDHINL KVAGQDGSVV QFKIKRHTPL
SKLMKAYCERQGLSMRQIRF RFDGQPINET DTPAQLEMED
EDTIDVFQQQ TGGVY AA MADEKPKEGV KTENNDHINL
KVAGQDGSVV QFKIKRHTPL SKLMKAYCER QGLSMRQIRF
RFDGQPINET DTPAQLEMED EDTIDVFQQQ TGGVY AA
MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AAT FFYGGCRGKRNNFKTEEY

SUMO2 immature form poly gene (3X)—(LRP-1/2) Angiopep-2 (SEQ ID no. 11):

MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AAMADEKPKEGV KTENNDHINL
KVAGQDGSVV QFKIKRHTPL SKLMKAYCERQGLSMRQIRF
RFDGQPINET DTPAQLEMED EDTIDVFQQQ TGGVY AA
MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA TFFYGGSRGKRNNFKTEEY

SUMO2 immature form poly gene (3X)—(LRP-1/2) Angiopep-5 3 (SEQ ID no. 12):

MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA MADEKPKEGV KTENNDHINL
KVAGQDGSVV QFKIKRHTPL SKLMKAYCER QGLSMRQIRF
RFDGQPINET DTPAQLEMED EDTIDVFQQQ TGGVY
AAMADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA TFFYGGSRGKRNNFRTEEY

SUMO2 immature form poly gene (3X)—the neuron-specific rabies viral glycoprotein (RVG29) peptide (SEQ ID no. 13):

MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA MADEKPKEGV KTENNDHINL
KVAGQDGSVV QFKIKRHTPL SKLMKAYCER QGLSMRQIRF
RFDGQPINET DTPAQLEMED EDTIDVFQQQ TGGVY
AAMADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA
YTIWMPENPRPGTPCDIFTNSRGKRASNG

SUMO2 immature form poly gene (2X)—Transferrin peptide (SEQ ID no. 26):

MADEKPKEGV KTENNDHINL KVAGQDGSVV QFKIKRHTPL
SKLMKAYCER QGLSMRQIRF RFDGQPINET DTPAQLEMED
EDTIDVFQQQ TGGVY AA MADEKPKEGV KTENNDHINL
KVAGQDGSVV QFKIKRHTPL SKLMKAYCER QGLSMRQIRF
RFDGQPINET DTPAQLEMED EDTIDVFQQQ TGGVY AA
CRTIGPSVC

It is further described an embodiment wherein SUMO mutants are used. Particularly preferred SUMO mutants are the following: SUMO2 K11A (SEQ ID no. 14), where lys 11 is substituted with ala to block polySUMOylation of the protein. SEQ ID no. 14:

MADEKPKEGV ATENNDHINL KVAGQDGSVV QFKIKRHTPL
SKLMKAYCER QGLSMRQIRF RFDGQPINET DTPAQLEMED
EDTIDVFQQQ TGGVY

SUMO2 Q90P (SEQ ID no. 15), where gln is substituted with pro to block deSUMOylation of the protein from targets. SEQ ID no. 15:

MADEKPKEGV KTENNDHINL KVAGQDGSVV QFKIKRHTPL
SKLMKAYCER QGLSMRQIRF RFDGQPINET DTPAQLEMED
EDTIDVFQQP TGGVY

In a further embodiment, pharmaceutical compositions are described, comprising one or more of the described SUMO proteins or fusion proteins, and a pharmaceutically acceptable carrier or excipient.

In a preferred embodiment, said SUMO proteins or fusion proteins are selected from the group comprising: SUMO2 immature form (SEQ ID no. 16), SUMO2 (SEQ ID no. 1), SUMO2 immature form—RVG29 (SEQ ID no. 7), SUMO2 immature form poly gene (3X)—Transferrin peptide (SEQ ID no. 8), or they are a SUMO variant having a sequence at least 80%, or at least 90%, or at least 95%, or at least 98%, or at least 99% identical to any one of said sequences: SEQ ID no. 16, SEQ ID no. 1, SEQ ID no. 7, SEQ ID no. 8.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, magnesium trisilicate, polyvinyl pyrrolidone, cellulose-based substances, polyacrylates, waxes, polyethylene glycol. The compositions of the present invention may be administered orally, parenterally, by inhalation spray, topically, rectally, nasally, buccally, vaginally or via an implanted reservoir. In a preferred embodiment, it is a pharmaceutical composition for parenteral administration. The term “parenteral” as used herein includes subcutaneous, intravenous, intramuscular, intra-articular, intra-synovial, intrasternal, intrathecal, intrahepatic, intralesional and intracranial injection or infusion techniques.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. The acceptable vehicles and solvents are preferably selected in the group comprising: water, Ringers solution and isotonic sodium chloride solution. In addition, sterile, fixed oils are conventionally employed as a solvent or suspending medium. For this purpose, any bland fixed oil may be employed including synthetic monoo- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspension may also contain a long-chain alcohol diluent or dispersant such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The pharmaceutical composition is here claimed for use in the treatment of neurodegenerative and/or neurological disorders, preferably in the treatment of Parkinson disease, Huntington disease, Alzheimer's disease and other tauopathies.

In a further embodiment, it is here claimed a method of treatment of neurodegenerative and/or neurological disorders, comprising administering to a subject in need thereof a pharmacologically active amount of a pharmaceutical composition according to the present description.

### EXPERIMENTAL SECTION

Example 1: in vitro test, the activity of SUMO2 (SEQ ID no. 1) versus tau-induced toxicity.

HEK 293 cells have been used as an experimental model. These cells have been transfected via lipofection with a plasmid encoding for human tau. As a control, cells were transfected with a pcDNA3 plasmid encoding GFP (Green Fluorescent Protein). At 0, 24 and 48 hours after transfection, 1 μg/ml of purified protein SUMO2 (SEQ ID no. 1) was added to the cell culture media. Cells viability was evaluated 48 and 72 hours after transfection.

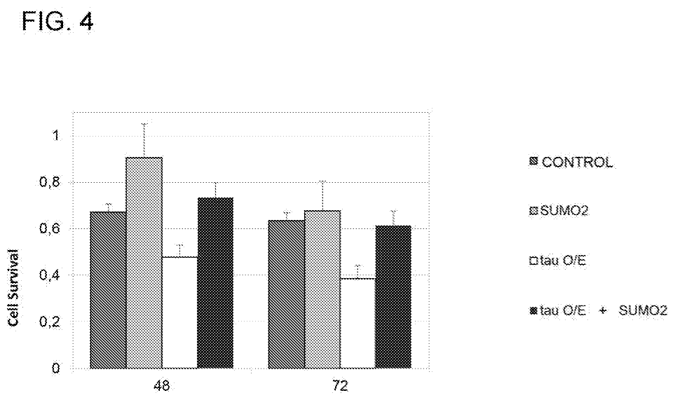

Results are reported in FIG. 4.

On the y axis arbitrary units show cell survival, two time points (48 h and 72 h after transfection) are shown.

The expression of tau induces toxicity in the cells, white column. The presence of SUMO2 (SEQ ID no. 1) per se, light grey column, is not toxic in the experimental model.

When tau is expressed in the presence of SUMO2 (SEQ ID no. 1), black column, a rescue from tau-induced toxicity is observed in the experimental models.

Data reported are the average results obtained from three independent experiments. In each one of the experiment, cell viability has been assessed in 10 quintuplicate.

Example 2: in vitro test, the activity of SUMO2 (SEQ ID no. 1), SUMO2 immature form (SEQ ID no. 7) and SUMO2 immature form poly gene (3X)—Transferrin peptide (SEQ ID no. 8) versus tau-induced toxicity.

HEK 293 cells have been used as an experimental model. These cells have been transfected via lipofection with a plasmid encoding for human tau. As a control, cells were transfected with a pcDNA3 plasmid encoding GFP (Green Fluorescent Protein). At 0 and 24 hours after transfection, 1 μg/ml of purified proteins SUMO2 (SEQ ID no. 1), SUMO2 immature form (SEQ ID no. 7) and SUMO2 immature form poly gene (3X)—Transferrin peptide (SEQ ID no. 8) was added to the cell culture media. Cells viability was evaluated 72 hours after transfection. Results are reported in FIG. 5. On the y axis arbitrary units show cell survival at one time point, 72h after transfection.

The expression of tau induces toxicity in the cells, white column. When tau is expressed in the presence of SUMO2 (SEQ ID no. 1), SUMO2 immature form (SEQ ID no. 7) and SUMO2 immature form poly gene (3X)—Transferrin peptide (SEQ ID no. 8) a rescue from tau-induced toxicity is observed in the experimental models. Data reported are the average results obtained from three independent experiments. In each one of the experiment, cell viability has been assessed in 10 quintuplicate.

Example 3: in vitro test, the activity of SUMO2 (SEQ ID no. 1), SUMO2 immature form (SEQ ID no. 7) and SUMO2 immature form poly gene (3X)—Transferrin peptide (SEQ ID no. 8) versus alpha-synuclein-induced toxicity.

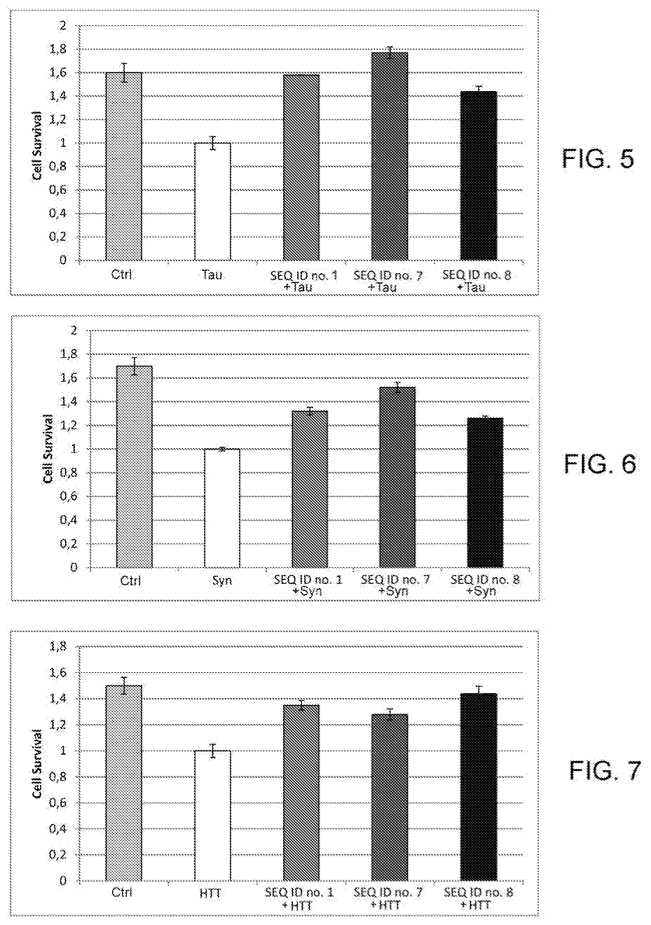

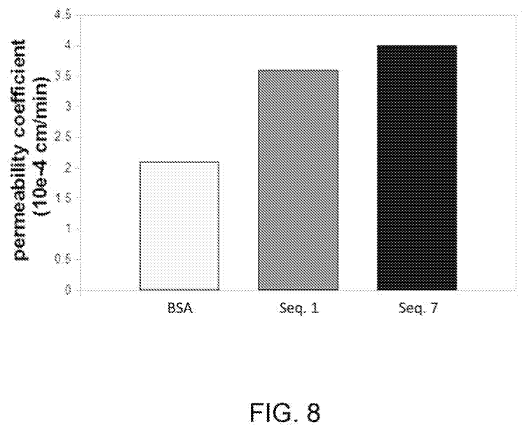

HEK 293 cells have been used as an experimental model. These cells have been transfected via lipofection with a plasmid encoding for human alpha-synuclein. As a control, cells were transfected with a pcDNA3 plasmid encoding GFP (Green Fluorescent Protein). At 0 and 24 hours after transfection, 1 μg/ml of purified proteins SUMO2 (SEQ ID no. 1), SUMO2 immature form (SEQ ID no. 7) and SUMO2 immature form poly gene (3X)—Transferrin peptide (SEQ ID no. 8) was added to the cell culture media. Cells viability was evaluated 72 hours after transfection. Results are reported in FIG. 6. On the y axis arbitrary units show cell survival at one time point, 72h after transfection.

The expression of alpha-synuclein induces toxicity in the cells, white column.

When alpha-synuclein is expressed in the presence of SUMO2 (SEQ ID no. 1), SUMO2 immature form (SEQ ID no. 7) and SUMO2 immature form poly gene (3X)—Transferrin peptide (SEQ ID no. 8) a rescue from alpha-synuclein-induced toxicity is observed in the experimental model.

Data reported are the average results obtained from three independent experiments. In each one of the experiment, cell viability has been assessed in 10 quintuplicate.

Example 4: in vitro test, the activity of SUMO2 (SEQ ID no. 1), SUMO2 immature form (SEQ ID no. 7) and SUMO2 immature form poly gene (3X)—Transferrin peptide (SEQ ID no. 8) versus HTT-induced toxicity.

HEK 293 cells have been used as an experimental model. These cells have been transfected via lipofection with a plasmid encoding for the N-terminal portion of mutant human huntingtin (HTT). As a control, cells were transfected with a pcDNA3 plasmid encoding GFP (Green Fluorescent Protein). At 0 and 24 hours after transfection, 1 μg/ml of purified proteins SUMO2 (SEQ ID no. 1), SUMO2 immature form (SEQ ID no. 7) and SUMO2 immature form poly gene (3X)—Transferrin peptide (SEQ ID no. 8) was added to the cell culture media. Cells viability was evaluated 72 hours after transfection. Results are reported in FIG. 7. On the y axis arbitrary units show cell survival. On the x axis, one time point (72h after transfection) is shown.

The expression of HTT induces toxicity in the cells, white column. When HTT is expressed in the presence of SUMO2 (SEQ ID no. 1), SUMO2 immature form (SEQ ID no. 7) and SUMO2 immature form poly gene (3X)—Transferrin peptide (SEQ ID no. 8) a rescue from HTT-induced toxicity is observed in the experimental models. Data reported are the average results obtained from three independent experiments. In each one of the experiment, cell viability has been assessed in 10 quintuplicate.

Example 5: blood brain barrier permeability of SUMO2 (SEQ ID no. 1) and SUMO2 immature form (SEQ ID no. 7).

Blood brain barrier (BBB) permeability was evaluated using a cell monolayer model comprised by cells from human temporal lobe microvessels isolated from tissue and the permeability marker bovine serum albumin (BSA). SUMO2 (SEQ ID no. 1), SUMO2 immature form (SEQ ID no. 7) or BSA were loaded on the luminal side of the two-chambers tissue culture system. Samples were removed from the abluminal chamber at 60, 90, and 120 min and then the concentration of proteins encoding sequences 1, 7 or BSA was determined. The permeability coefficients were calculated according to the method described by Dehouck et al. (1992).

SUMO2 (SEQ ID no. 1) and SUMO2 immature form (SEQ ID no. 7) show good permeability of the BBB.

1. A protein for treating neurodegenerative disorders and/or neurological disorders, the protein comprising a SUMO protein, or a variant or a fragment thereof, or a fusion protein comprising at least one SUMO protein, or a variant or a fragment thereof.

2. The protein or fusion protein for use according to claim 1, wherein said SUMO protein is a SUMO isoform selected from the group consisting of: SUMO1 immature form (SEQ ID no. 20), SUMO2 immature form (SEQ ID no. 16), SUMO3 immature form (SEQ ID no. 21), SUMO4 immature form (SEQ ID no. 22), SUMO1 (SEQ ID no. 23), SUMO2 (SEQ ID no. 1), SUMO3 (SEQ ID no. 24), SUMO4 (SEQ ID no. 25), or said SUMO protein is a SUMO mutant selected from SUMO2 K11A (SEQ ID no. 14), SUMO2 Q90P (SEQ ID no. 15) or said SUMO protein is a SUMO variant having a sequence at least 80%, or at least 90%, or at least 95%, or at least 98%, or at least 99% identical to any one of said sequences: SEQ ID no. 20, SEQ ID no. 21, SEQ ID no. 22, SEQ ID no. 23, SEQ ID no. 24, SEQ ID no. 25, SEQ ID no. 1, SEQ ID no. 16, SEQ ID no. 14, SEQ ID no. 15.

3. The protein or fusion protein according to claim 2, wherein said SUMO protein is SUMO2 in the immature form defined by SEQ ID no. 16.

4. The protein or fusion protein according to claim 1, wherein said protein is a fusion protein and further comprises a carrier linked at said SUMO protein C-terminus, wherein said carrier is selected among the group comprising: peptide linking the transferrin receptor, apolipoprotein B (apoB), (LRP-1/2) Angiopep-1, (LRP-1/2) Angiopep-2, (LRP-1/2) Angiopep-3, Rabies Virus Glycoprotein 29 (RVG29).

5. The protein or fusion protein according to claim 4, wherein said carrier is linked to said SUMO protein C-terminus via a linker, said linker being any short amino acid sequence, said linker being selected from the group comprising the amino acidic sequences: AA, (GGGG)n where n indicates that said sequence GGGG (SEQ ID no. 17) is repeated at least once in said linker, (GGGGS)n where n indicates that said sequence GGGGS (SEQ ID no. 18) is repeated at least once in said linker, more.

6. The protein or fusion protein according to claim 1, wherein said protein is a fusion protein and further comprises at least a second SUMO protein, said at least second SUMO protein being selected from the group comprising: SUMO2 in the immature form, defined by SEQ ID no. 16, or in the mature form, defined by SEQ ID no. 1, or a variant thereof having a sequence at least 80% identical to SEQ ID no. 16 or to SEQ ID no. 1.

7. The protein or fusion protein according to claim 6, wherein said at least two SUMO proteins are linked together by a linker linking the C-terminus of said first SUMO protein in the immature form with the N-terminus of said at least second SUMO protein, said linker being any short amino acidic sequences, said linker selected from the group comprising the amino acidic sequences: AA, (GGGG)n where n indicates that said sequence GGGG (SEQ ID no. 17) is repeated at least once in said linker, (GGGGS)n where n indicates that said sequence GGGGS (SEQ ID no. 18) is repeated at least once in said linker.

8. The protein or fusion protein according to claim 1, said protein being a fusion protein selected from the group consisting of: SUMO2 immature form—transferrin peptide (SEQ ID no. 2), SUMO2 immature form—ApoB (SEQ ID no. 3), SUMO2 immature form—(LRP-1/2) Angiopep-1 (SEQ ID no. 4), SUMO2 immature form—(LRP-1/2) Angiopep-2 (SEQ ID no. 5), SUMO2 immature form—(LRP-1/2) Angiopep-3 (SEQ ID no. 6), SUMO2 immature form—RVG29 (SEQ ID no. 7), SUMO2 immature form poly gene (3X)—Transferrin peptide (SEQ ID no. 8), SUMO2 immature form poly gene (3X)—ApoB (SEQ ID no. 9), SUMO2 immature form poly gene (3X)—(LRP-1/2) Angiopep-1 (SEQ ID no. 10), SUMO2 immature form poly gene (3X)—(LRP-1/2) Angiopep-2 (SEQ ID no. 11), SUMO2 immature form poly gene (3X)—(LRP-1/2) Angiopep-3 (SEQ ID no. 12), SUMO2 immature form poly gene (3X)—RVG29 (SEQ ID no. 13), SUMO2 immature form poly gene (2X)—Transferrin peptide (SEQ ID no. 26).

9. The protein or fusion protein according to claim 1, wherein said SUMO protein is SUMO2.

10. The protein or fusion protein according to claim 9, said protein being selected from the group consisting of: SUMO2 (SEQ ID no. 1), SUMO2 immature form (SEQ ID no. 7) and SUMO2 immature form poly gene (3X)—Transferrin peptide (SEQ ID no. 8).

11. The protein or fusion protein according to claim 1, wherein said neurodegenerative and/or neurological disorders are selected from Parkinson disease, Huntington disease, Alzheimer's disease and other tauopathies.

12. A pharmaceutical composition for treating neurodegenerative disorders and/or neurological disorders comprising at least one of the proteins or fusion proteins according to claim 1.

13. The pharmaceutical composition according to claim 12, further comprising carrier or excipients for oral, parental, inhalatory, topical, rectal, nasal, oral, vaginal administration, or via an implant, for parental administration.

14. A pharmaceutical preparation comprising a pharmacologically active amount of at least one of the SUMO proteins or fusion proteins and, one or more active principle for use in the treatment of neurodegenerative and/or neurological disorders.

15. The pharmaceutical composition for use according to claim 14, wherein said at least one SUMO is selected from the group comprising: SUMO2 immature form (SEQ ID no. 16), SUMO2 (SEQ ID no. 1), SUMO2 immature form—RVG29 (SEQ ID no. 7), SUMO2 immature form poly gene (3X)—Transferrin peptide (SEQ ID no. 8), or they are a SUMO variant having a sequence at least 80%, or at least 90%, or at least 95%, or at least 98%, or at least 99% identical to any one of said sequences: SEQ ID no. 16, SEQ ID no. 1, SEQ ID no. 7, SEQ ID no. 8.

16. The pharmaceutical composition according to claim 13, wherein said use is in reducing, inhibiting toxic oligomers or aggregates or removing and/or preventing formation of toxic oligomers or aggregates in a patient.

17. A method to reduce or prevent formation of toxic aggregates in a patient, said method comprising administering to the patient an effective amount of a pharmaceutical preparation comprising at least one of the proteins or fusion proteins according to claim 1.

18. A method of treatment of neurodegenerative and/or neurological disorders, comprising administering to a subject a pharmacologically active amount of a pharmaceutical composition according to claim 13.


SEQUENCE LISTING (26 sequences)

SEQ ID NO: 1 - protein, 95 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 2 - protein, 106 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAACRTIGPSVC

SEQ ID NO: 3 - protein, 136 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAASSVIDALQYKLEGTTRLTRKRGLKLATALSLSNKFVEGS

SEQ ID NO: 4 - protein, 116 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGCRGKRNNFKTEEY

SEQ ID NO: 5 - protein, 116 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGSRGKRNNFKTEEY

SEQ ID NO: 6 - protein, 116 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGSRGKRNNFRTEEY

SEQ ID NO: 7 - protein, 126 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAYTIWMPENPRPGTPCDIFTNSRGKRASNG

SEQ ID NO: 8 - protein, 300 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAACRTIGPSVC

SEQ ID NO: 9 - protein, 330 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAASSVIDALQYKLEGTTRLTRKRGLKLATALSLSNKFVEGS

SEQ ID NO: 10 - protein, 310 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGCRGKRNNFKTEEY

SEQ ID NO: 11 - protein, 310 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGSRGKRNNFKTEEY

SEQ ID NO: 12 - protein, 310 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAATFFYGGSRGKRNNFRTEEY

SEQ ID NO: 13 - protein, 320 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAYTIWMPENPRPGTPCDIFTNSRGKRASNG

SEQ ID NO: 14 - protein, 95 aa, Homo sapiens
MADEKPKEGVATENNDHINLKVAGQDGSVVQFKIKRHTPLSKLMKAYCERQGLSMRQIRFRFDGQPINETDTPAQLEMEDEDTIDVFQQQTGGVY

SEQ ID NO: 15 - protein, 95 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQPTGGVY

SEQ ID NO: 16 - protein, 97 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAA

SEQ ID NO: 17 - protein, 4 aa, Homo sapiens
GGGG

SEQ ID NO: 18 - protein, 5 aa, Homo sapiens
GGGGS

SEQ ID NO: 19 - protein, 15 aa, Homo sapiens
GGGGSGGGGSGGGGS

SEQ ID NO: 20 - protein, 101 aa, Homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 21 - protein, 103 aa, Homo sapiens
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 22 - protein, 95 aa, Homo sapiens
MANEKPTEEVKTENNNHINLKVAGQDGSVVQFKIKRQTPLSKLMKAYCEPRGLSMKQIRFRFGGQPISGTDKPAQLEMEDEDTIDVFQQPTGGVY

SEQ ID NO: 23 - protein, 97 aa, Homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 24 - protein, 92 aa, Homo sapiens
MSEEKPKEGVKTENDHINLKVAGQDGSVVQFKIKRHTPLSKLMKAYCERQGLSMRQIRFRFDGQPINETDTPAQLEMEDEDTIDVFQQQTGG

SEQ ID NO: 25 - protein, 93 aa, Homo sapiens
MANEKPTEEVKTENNNHINLKVAGQDGSVVQFKIKRQTPLSKLMKAYCEPRGLSMKQIRFRFGGQPISGTDKPAQLEMEDEDTIDVFQQPTGG

SEQ ID NO: 26 - protein, 203 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVYAAMADEKPKEGVKTENNDHINLKVAGQDGSVVQFKIKRHTPLSKLMKAYCERQGLSMRQIRFRFDGQPINETDTPAQLEMEDEDTIDVFQQQTGGVYAACRTIGPSVC